(19) United States
(12) Patent Application Publication
(10) Pub. No.: US 2017/0261513 A1
(43) Pub. Date: Sep. 14, 2017

(54) MONOCLONAL ANTIBODY BASED ONLINE PHOSPHOPROTEIN PROTEOMICS ANALYSIS METHOD USING MICROBORE HOLLOW FIBER ENZYMATIC REACTOR-TANDEM MASS SPECTROMETRY

(71) Applicant: KOREA RESEARCH INSTITUTE OF STANDARDS AND SCIENCE, Daejeon (KR)

(72) Inventors: Dukjin KANG, Daejeon (KR); Sun Young LEE, Daejeon (KR); Sook-Kyung KIM, Daejeon (KR)

(21) Appl. No.: 15/504,732

(22) PCT Filed: Aug. 19, 2015
(86) PCT No.: PCT/KR2015/008641

§ 371 Date: Mar. 22, 2017

(30) Foreign Application Priority Data
Aug. 19, 2014 (KR) 10-2014-0107564

(51) Int. Cl. G01N 33/68; H01J 49/00

(52) U.S. Cl. CPC G01N 33/6848; H01J 49/0027; H01J 49/004

(57) ABSTRACT

A phosphoprotein extraction method and a mass spectrometric method using a microbore hollow fiber enzymatic reactor (mHFER) based antigen-antibody reaction and, specifically, to an extraction method and a mass spectrometric method, wherein phosphoproteins or phosphopeptides present in the body are extracted using phosphoserine-, phosphothreonine-, and phosphotyrosine-antibodies, and measured by a mass spectrometer, and thus biomarker phosphoproteins for diagnosis of diseases are found, contributing to early diagnosis of diseases. The mass spectrometric method using the antigen-antibody reaction based extraction method can: minimize temporal and economic burdens resulting from a low extraction rate and a complicated sample pre-treatment; increase the extraction efficiency by using a considerable number of phosphopeptides (or phosphoproteins) and antibodies with strong affinity; and allow the extraction of low-concentration phosphopeptides or phosphoproteins, and thus is expected to have high applicability in discovering disease diagnosis protein markers and identifying and studying mechanisms thereof.

### TECHNICAL FIELD

The present invention relates to development of a mHFER-based online phosphopeptide-specific pretreatment method using antibodies with a specific affinity to serine-phosphate, threonine-phosphate and tyrosine-phosphate groups among post-translational modifications (PTMs) of protein, and an analysis technique for qualitative analysis of phosphorylated protein in a biological sample and even confirmation of phosphorylation sites in protein using a tandem mass spectrometer, from phosphopeptides collected through the online pretreatment process.

### BACKGROUND ART

A study of proteomics based on a mass spectrometer plays an important role in structure identification and quantitative analysis of protein, and is used as a means for understanding a gene function. In order to diagnose human diseases, protein present in various and complicated biological samples obtainable from human beings is subjected to qualitative and quantitative analysis. Generally, protein PTMs are chemical modifications and are involved in interaction and activity regulation between protein, nucleic acid, lipid and other cell molecules such as cofactor, and thus, play a key role in a functional aspect of proteome. In particular, phosphoprotein is known to affect interaction between cells, apoptosis and cytogenesis, and on/off of a protein function. In addition, most phosphorylation is distributed in serine in an amino acid sequence of protein a lot, and also it is known that phosphorylation occurs also in threonine and tyrosine. Phosphoprotein exists in a relatively small amount as compared with other proteins present in the human body, and particularly, has relatively low ESI efficiency as compared with peptides produced by a general enzymatic treatment process, when being subjected to proteomics analysis using a cation mode-based electrospray ionization-tandem mass spectrometer (ESI-MS/MS), due to a negative charge of a phosphate group, and as a result, has difficulty in qualitative and quantitative analysis of phosphopeptides. For the above reasons, various pretreatment methods are developed and applied, in order to increase qualitative analysis efficiency based on a conventional mass spectrometer for phosphoproteome or phosphopeptides. As a conventional selective pretreatment method for phosphoproteome or phosphopeptides, it was suggested that peptides are separated using hydrophilic interaction liquid chromatography (HILIC), strong anion-exchange chromatography (SAX), strong cationic ion-exchange chromatography (SCX) and the like, subjected to pretreatment such as immobilized metal affinity chromatography (IMAC) and metal oxide affinity chromatography (MOAC) using a chemical bond between a positive charge of metal ions and a negative charge of a phosphate group to extract only phosphoprotein, and then introduced to a mass spectrometer. As analysis by an immunological method, 2 dimensional gel electrophoresis (2D GE), immunoblot, immunoprecipitation (IP) and the like are used. Particularly, immunoblot and immunoprecipitation are the methods for identifying targeted protein in gel using a binding principle between an antibody and an antigen, and in the case of using the methods, it is advantageous to extract only the protein to be effectively analyzed. However, since antibodies are not all coated on silanol groups of a membrane or beads used in the methods, undesired protein is extracted together to lower extraction reproducibility, and thus, in order to prevent this, other pretreatment processes such as treating the silanol group not coated with the antibody with albumin are necessarily required. In addition, when introducing the extracted protein to the mass spectrometer, it should be subjected to a secondary pretreatment process (removal of a surfactant used when extraction, or peptidization of protein), and thus, there occurs a sample loss due to a complicated pretreatment.

### DISCLOSURE

### Technical Problem

An object of the present invention is to provide an online antibody-specific phosphoprotein mass spectrometric method using an antigen-antibody reaction based on a microbore hollow fiber membrane enzymatic reactor, which may improve a complicated sample pretreatment process and low extraction efficiency of a method using an affinity between phosphopeptide or phosphoprotein and metal ions.

### Technical Solution

In one general aspect, a mass spectrometric method includes a) adding a reducing agent to a protein mixture present in cells to perform denaturation, and then carrying out a reaction with an enzyme to obtain a peptide mixture; b) binding the peptide mixture from step a) to a phosphoprotein or phosphopeptide-specific antibody; and c) extracting phosphopeptides obtained by injecting a reactant obtained in step b) to a microbore hollow fiber membrane enzymatic reactor (mHFER) and being subjected to enzymatic treatment, thereby obtaining a mass spectrum.

The protein mixture used in above step a) is not limited, but a cell lysate extracted from cells may be used. In the present invention, the reducing agent is not limited, but dithiothreitol (DTT), dithioerythritol, tris 2-carboxyethyl phosphine, tributyl phosphine or the like may be used, and preferably dithiothreitol (DTT) may be used to proceed with denaturation of protein.

The enzyme is not limited, but any enzyme may be used, as long as it is a protease, and preferably trypsin may be used.

After the denaturation, in order to prevent refolding of a disulfide bond between cysteine-cysteine in protein, iodoacetamide (IAA) is added to alkylate a thiol group (−SH) of cysteine, thereby performing deformation into an irreversible form. Since remaining IAA may induce deformation or an additional reaction by light, L-cysteine is added thereto to remove IAA. The solution may be added so that a weight ratio between trypsin and protein is 1:40 to 1:60, thereby carrying out the reaction in a temperature agitator at 37° C. for 18 hours to perform enzymatic treatment.

In above step b), the phosphopeptide or phosphoprotein-specific antibody is not limited, but any one antibody or an antibody mixture of two or more selected from the group consisting of phosphoserine-, phosphothreonine- and phosphotyrosine-antibodies having a molecular weight of 50 kDa or more may be used. The phosphoserine-antibody has an affinity to a serine-phosphate group, the phosphothreonine-antibody has an affinity to a threonine-phosphate group, and the phosphotyrosine-antibody has an affinity to a tyrosine-phosphate group, respectively. A mixing ratio of the phosphopeptide or phosphoprotein and the antibody is not limited, but the phosphoprotein or the mixture of phosphoprotein may be at 10 to 1000 parts by weight based on 100 parts by weight of the antibody or antibody mixture, and bonds between serine-, threonine- and tyrosine-phosphate groups and each antibody are formed by the reaction.

The hollow fiber membrane used in above step c) is not limited, but the hollow fiber membrane having a molecular weight permeation limit value of 10 kDa, an inner diameter of about 200 to 600 μm, and an outer diameter of about 500 to 1000 μm is preferred, and it is preferred that the materials thereof consist of polystyrene sulfonate, polyvinyl chloride, polyacrylonitrile, a mixture thereof or the like. The extraction is not limited, but may be carried out at 4 to 25° C., and when carrying out extraction within the above range through the exemplary embodiment of the present invention, a number of phosphopeptides may be obtained. When injecting the antibody-binding peptide mixture obtained from above step b) to a microbore hollow fiber membrane enzymatic reactor (mHFER), the antibody-binding peptide having a size of 10 kDa or more stays in the mHFER, and the antibody-unbinding peptide having a molecular weight less than 10 kDa escapes from the mHFER to be eluted. In the reaction, a protease such as trypsin is injected to decompose the antibody to lose the function thereof, which allows the peptide binding to the antibody to be eluted. The peptide is collected in a reverse trapping column and separated with a reverse phase C18 column which is a separation means depending on a hydrophobicity degree, and thereafter, is subjected to electrospray ionization (ESI), and introduced to a mass spectrometer and analyzed. The mass spectrometer in above step c) is not limited, but for example, nano-flow rate liquid chromatography (nLC, 1260 capillary LC system, Agilent Technologies, Germany)-electrospray ionization-Fourier transformation orbitrap tandem mass spectrometer (ESI-FT orbitrap-MS/MS, Q-Exactive, Thermo Scientific, Germany) may be used.

### Advantageous Effects

The online phosphopeptide or phosphoprotein extraction method according to the present invention has merits of efficiently extracting antibody-specific phosphopeptides from a peptide mixture produced by enzymatic treatment, and also minimizing the problems arising in the conventional complicated pretreatment process by an automated system configuration using a mHFER device, by using an antibody having a specific affinity to phosphorylated peptide present in a relatively low concentration.

### DESCRIPTION OF DRAWINGS

FIG. 1 is a schematic view for a phosphoproteome extraction method using an antigen-antibody reaction based on a microbore hollow fiber membrane enzymatic reactor (mHFER) according to the present invention.

FIG. 2 is a base peak chromatogram and a mass spectrum obtained by subjecting protein obtained by using a cell sample (MCF7) to enzymatic treatment, and then reacting a peptide mixture with an antibody, and performing measurement with hollow fiber membrane enzymatic reactor-based online nLC-ESI-FT orbitrap-MS/MS.

FIG. 3 is the number of phosphopeptides measured by a ratio of protein to 10 μg of antibody, when extracting antibody-binding phosphopeptide or phosphoprotein.

FIG. 4 is the number of phosphopeptides measured depending on temperature of the reaction between phosphopeptide or phosphoprotein and an antibody for the same case as FIG. 3.

FIG. 5 is the number of phosphopeptides measured by reacting phosphoserine-antibody, phosphothreonine-antibody and phosphotyrosine-antibody, respectively, according to the established condition.

FIG. 6 is the number of phosphopeptides extracted by the extraction method according to the present invention and the conventional phosphoproteome extraction method (FASP, IMAC, TiO₂) from a peptide mixture (10 μg) obtained from a MCF7 cell lysate.

FIG. 7 is the result obtained from FIG. 6, which is the number of phosphorylation sites present in serine, threonine and tyrosine in extracted phosphopeptides.

### BEST MODE

Hereinafter, the present invention will be described in detail with reference to the Examples and accompanying drawings. However, they are for describing the present invention in more detail, and the scope of the present invention is not limited to the following Examples.

### Experimental Example 1

### Extraction of Phosphopeptides Produced from Protein Present in Cell Lysate

MCF7 (Korean Cell Line Bank, Republic of Korea) cells (5*10⁶/10 cm dish) were collected, added to 0.1 M PBS (phosphate buffered saline), and subjected to ultrasonic fragmentation using a tip sonicator, and then centrifuged at 10,000 rpm for 10 minutes. The supernatant was separated, and an aliquot of 100 μg of protein extracted therefrom was mixed with a 50 mM ammonium bicarbonate solution and a 10 mM solution with DTT added, and then denaturation was performed at 37° C. for 2 hours. To the solution, 32 μL of a 270 mM IAA solution was added, alkylation was performed in a dark chamber at room temperature for 30 minutes, and then 47 μL of 400 mM L-cysteine was added to remove remaining IAA. Thereafter, 2 μg of trypsin was added and peptidization was performed at 37° C. for 18 hours. The thus-produced peptides and a mixture of phosphoserine-antibody, phosphothreonine-antibody and phosphotyrosine-antibody were mixed and reacted under the composition and temperature conditions of the following Table 1, thereby preparing an antibody-binding peptide mixture.

### Experimental Example 2

### Phosphopeptide Mass Spectrometric Method

In the online microbore hollow fiber membrane enzymatic reactor (mHFER), a pump which is adjustable to a flow rate of 1 to 10 μL/min or less and a sample injector (or autosampler) with which a sample is online injectable are connected to an inlet of the hollow fiber membrane, for transfer and injection of the antibody-binding peptide or enzyme. One side of the microbore hollow fiber membrane (mHF) used in the mHFER is blocked using epoxy so that the flowing path of flow passes only in the inner wall, and the permeation limit of the mHF is 10 kDa, having a volume of about 5 μL.

After injecting the antibody-binding peptide mixture prepared in Experimental Example 1 to the mHFER, 0.1 M PBS was flowed at a flow rate of 1 to 5 μL/min for 30 minutes to 1 hour. After removing peptide having a size less than 10 kDa, unbound to antibody in the mixture via the process, a reverse phase trapping column was installed on the outlet of the hollow fiber membrane. Trypsin was injected into the mHFER, and 0.1 M PBS was flowed at a flow rate of 1 to 5 μL/min for 30 minutes to 1 hour.

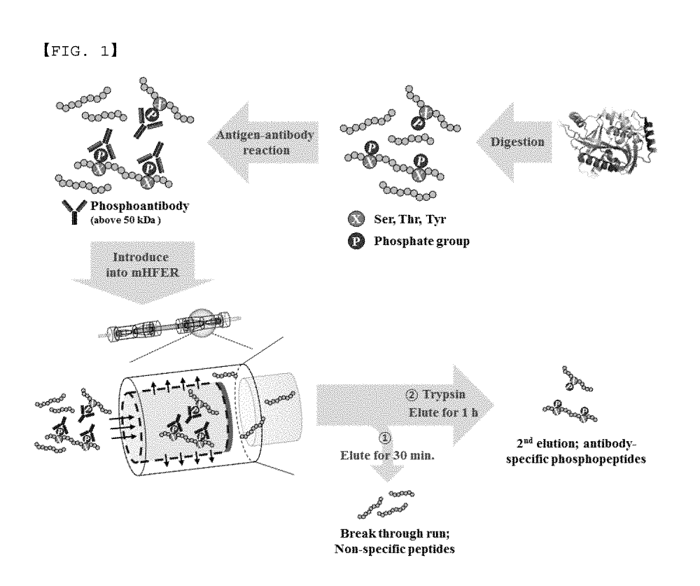

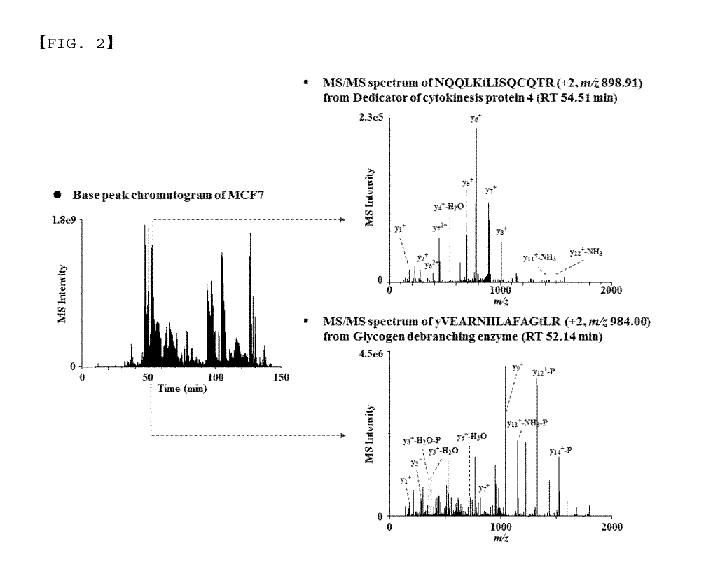

Decomposition of antibodies was caused by the reaction of antibody-binding phosphopeptide of 10 kDa or more collected in the mHFER and trypsin, so that the phosphopeptides which were bound to antibodies were separated, and eluted. The eluted phosphopeptides were collected in the reverse phase column connected to the outlet the mHFER, directly connected to a flow path of an instrument of nanoLC-ESI-FT orbitrap-MS/MS, eluted depending on a hydrophobicity degree of the phosphopeptides through a column filled with C18 according to a reverse phase solvent gradient by a binary pump, and introduced to the mass spectrometer. The series of processes was schematized in FIG. 1, and the example of the result of mass spectrometry was schematized in FIG. 2.

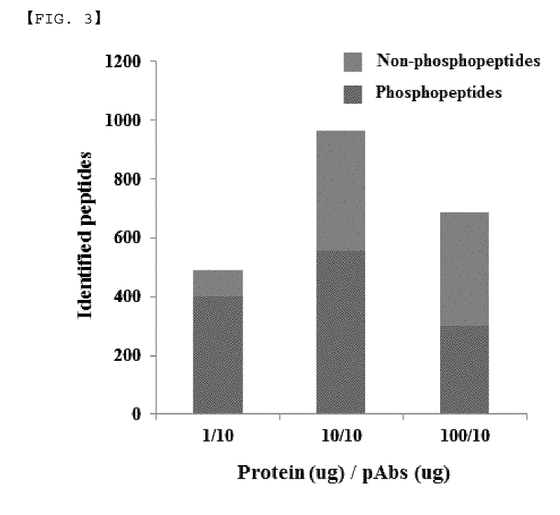

Examples 1 to 3 relate to the number of extracted phosphoprotein and phosphopeptide depending on the weight ratio between protein or peptide and an antibody, and according to the result of FIG. 3, it was shown that when the weight ratio between peptide and an antibody is 1:10, 1:1 and 10:1, the measured number of phosphopeptide was 400, 553 and 229, respectively, and it was confirmed therefrom that the number of extracted phosphopeptide was highest when the weight ratio was 1:1.

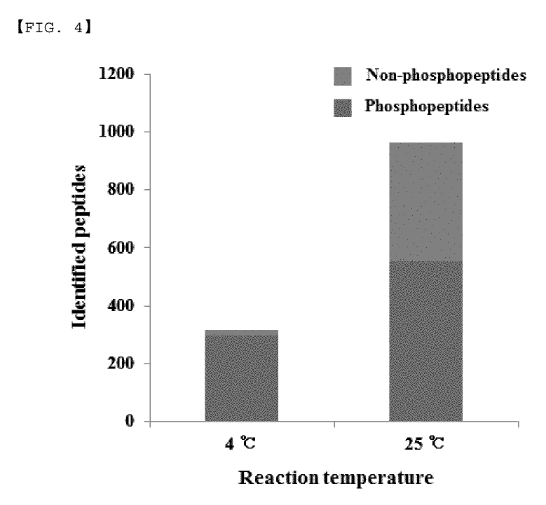

As a result of measuring extraction efficiency under the condition of Examples 2 and 4, when reaction temperature was 25° C. as in FIG. 4, 553 phosphopeptides were identified, and when reaction temperature was 4° C., 296 phosphopeptides were identified. Therefore, it was confirmed that when reaction temperature was 25° C., the number of extracted phosphopeptides was increased.

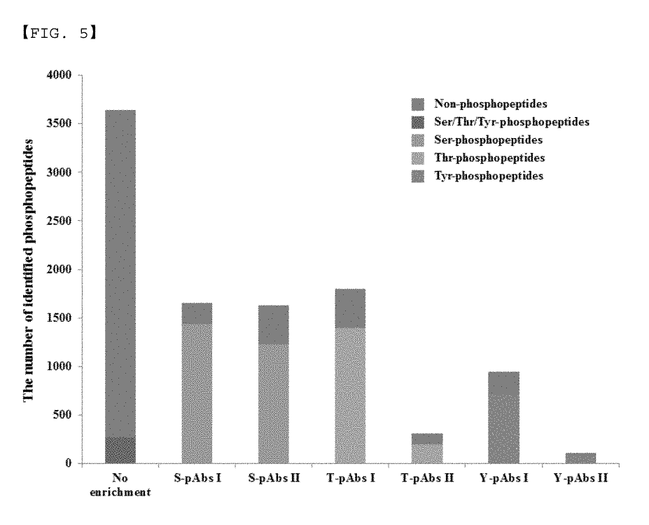

Examples 5 to 7 are the results of comparison of the reaction of protein or peptide and each antibody (using 2 antibodies from different sources, respectively, in phosphoserine-antibody, phosphothreonine-antibody, and phosphotyrosine-antibody), and as shown in FIG. 5, the number of phosphopeptides measured before extraction was 272, and the number of phosphopeptides measured using phosphoserine-antibodies I and II was 1,438 and 1,229, respectively. Further, the number of phosphothreonine-antibodies I and II was measured as 1,397 and 202, and the number of phosphopeptides extracted by phosphotyrosine-antibodies I and II was confirmed to be 713 and 76, respectively. Based on these results, it was confirmed that the number of phosphopeptides measured when using each antibody was increased as compared with that of an unextracted one, and particularly, the measured number of phosphoserine-peptide was highest, and the measured number was high in an order of phosphothreonine-peptide and phosphotyrosine-peptide.

### Experimental Example 3

### Efficiency Measurement According to Phosphopeptide Extraction

After enzyme-treating the protein obtained from the MCF7 cell lysate, the extraction efficiency of phosphoprotein by the previously reported extraction method of phosphopeptide [filter aided sample preparation (FASP), immobilized metal affinity chromatography (IMAC), titanium dioxide (TiO₂)] and that by the present invention were compared, using 10 μg of the peptide mixture.

FASP Extraction Method

In FASP, 10 μg of the peptide mixture and 10 μg of antibodies were reacted, and transferred to a centrifugal filter having a permeation limit of 10 kDa, and 200 μL of 0.1 M PBS was added and mixing was carried out, and then centrifugation was carried out at 14,000×g for 10 minutes. This process was repeated twice to remove the peptides unbound to antibodies, and 0.2 μg of trypsin (antibody:enzyme=50:1, w/w) was added and reacted at 37° C. for 18 hours. 200 μL of 0.1 M PBS was added to the enzyme-treated mixture and mixed, and then centrifuged at 14,000×g for 10 minutes to collect phosphopeptides separated from the antibodies. The process was further repeated once more to collect phosphopeptides, and the collected solution was concentrated using a vacuum concentrator, and then introduced to the mass spectrometer, and measurement was performed.

IMAC Extraction Method

For extraction of phosphopeptides using IMAC, a Ni-NTA spin column (Qiagen, Hilden, Germany) was used. The Ni-NTA beads were used by being filled into the capillary having one end blocked by porous glass sol-gel frits of 3 mm (inner diameter 200 um, length 100 mm). Installation was performed by connecting a syringe pump, a sample injector and the capillary filled with Ni-NTA beads in this order, and flowing the solvent at a flow rate of 1 to 5 μL/min to introduce the sample thereto. 100 μL of a 50 mM EDTA solution dissolved in a 0.1 M NaCl solution was flowed to remove Ni²⁺ ions present in the Ni-NTA beads, and 100 μL of a 0.2 M FeCl₃ solution was flowed to add Fe³⁺ to the NTA beads to be activated. Before injecting a sample, a loading buffer (0.1 M NaOH solution containing 6% acetic acid, pH 3.6) was flowed into the capillary for 30 minutes to equilibrate the NTA beads. 10 μg of the peptide mixture was injected through the sample injector, and then the loading buffer was flowed for 20 minutes, so that phosphoprotein or phosphopeptides are chelated to the Fe-NTA beads. In order to remove unchelated peptides, a washing buffer (loading buffer/acetonitrile, 75/25, v/v) was flowed into the capillary to remove the unchelated peptides from the capillary. Before lysing the chelated phosphoprotein, 100 μL of the loading buffer was flowed to perform equilibrium, and a 4% NH₄OH solution at pH 11 was flowed to collect the chelated peptides by elution. The collected solution was dried using a vacuum concentrator, and then redissolved in a 0.1% formic acid, and measurement was performed by introducing the solution to the mass spectrometer.

TiO₂ Extraction Method

For extraction of phosphopeptides using TiO₂, a solid phase extraction (SPE) cartridge (GL Science, Japan, 50 mg/3 mL) filled with TiO₂ beads was used. The cartridge was disposed in a 15 mL tube so as to use the centrifuge. To activate the dried SPE cartridge, 200 μL of buffer A containing 0.5% trifluoroacetic acid (acetonitrile/water=80/20, v/v) was added, centrifugation was performed at 200×g for 2 minutes, 200 μL of buffer B (lactic acid/buffer A=300 mg/mL, w/v) was added, centrifugation was performed at 200×g for 2 minutes, and the solution collected in the tube was discarded. 10 μg of the peptide mixture was added to the TiO₂ cartridge, 1000 μL of buffer B per 500 μL of the sample was mixed therewith in the cartridge, and then centrifugation was performed at 200×g for 2 minutes. The solution collected in the tube was added to the TiO₂ cartridge again, centrifugation was performed under the same condition, and the process was repeated 10 times, thereby chelating more phosphopeptides in the TiO₂ beads. In order to remove peptides other than the phosphopeptides in the TiO₂ cartridge, centrifugation was performed at 200×g for 2 minutes using 200 μL of buffer B, and at 200×g for 2 minutes with 200 μL of buffer A, and washing was performed. In order to elute the phosphopeptides, the TiO₂ cartridge was transferred to a new 15 mL tube, centrifugation was performed at 200×g for 5 minutes using 200 μL of a 5% aqueous ammonium solution, and under the same condition using 200 μL of a 5% aqueous pyrrolidine solution, thereby eluting the phosphopeptides, and then the collected two solutions were mixed and dried with the vacuum concentrator. The dried sample was redissolved in a 0.1% formic acid, and measurement was performed by introducing it to a mass spectrometer.

Results of Comparison of Conventional Three Extractions with Method Using mHFER

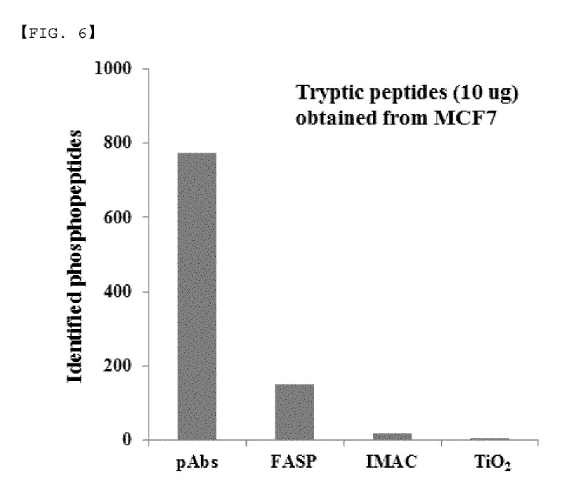

FIG. 6 represents the number of phosphopeptides extracted by the above-described three extractions and the present invention, wherein when the experiment was carried out using 10 μg of identical peptides, it was shown that 773 phosphopeptides were extracted by the method using the antibody mixture, 149 phosphopeptides by FASP, 18 phosphopeptides by IMAC, 2 phosphopeptides by TiO₂, and surprisingly, it was confirmed that phosphopeptides were extracted 5 times more when using the antibody mixture, than when using FASP, the conventional extraction method.

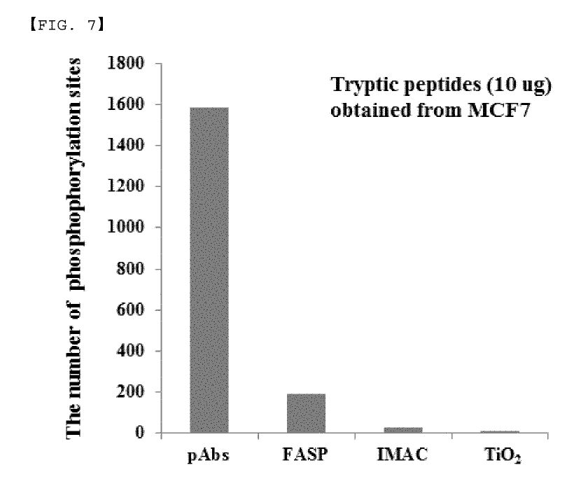

FIG. 7 represents the number of phosphorylation present in serine, threonine and tyrosine sites in the phosphopeptides through the experiments of FIG. 6. It was shown that there were 1,585 phosphorylation sites by the method using the antibody mixture, 189 phosphorylation sites by FASP, 24 phosphorylation sites by IMAC, and 2 phosphorylation sites by TiO₂ in the phosphopeptides. From the above results, it was confirmed that surprisingly, phosphorylation sites were 8 times more when using the extraction method using the antibody mixture than when using FASP, the conventional method.

Further, comparing the number of phosphopeptides in FIG. 6 with the number of phosphorylation of FIG. 7, it was confirmed that not only monophosphopeptides, but also multi phosphopeptides including di- and triphosphopeptides which were not able to be confirmed in the past were able to be extracted when using the antibody mixture extraction method of the present invention, thereby completing the present invention.

1. A mass spectrometric method comprising:
a) adding a reducing agent to a protein mixture present in cells to perform denaturation, and then carrying out a reaction with an enzyme to obtain a peptide mixture;
b) binding the peptide mixture from a) to a phosphoprotein or phosphopeptide-specific antibody; and
c) extracting phosphopeptides obtained by injecting a reactant obtained in b) to a microbore hollow fiber membrane enzymatic reactor (mHFER) and being subjected to enzymatic treatment, thereby obtaining a mass spectrum.

2. The mass spectrometric method of claim 1, wherein the reducing agent is dithiothreitol (DTT), dithioerythritol, tris 2-carboxyethyl phosphine or tributyl phosphine.

3. The mass spectrometric method of claim 1, wherein the enzyme is a protease.

4. The mass spectrometric method of claim 3, wherein the protease is trypsin.

5. The mass spectrometric method of claim 1, wherein the phosphoprotein or phosphopeptide-specific antibody is any one antibody or an antibody mixture of two or more selected from the group consisting of phosphoserine-, phosphothreonine- and phosphotyrosine-antibodies.

6. The mass spectrometric method of claim 1, wherein the phosphoprotein or phosphoprotein mixture is at 10 to 1000 parts by weight, based on 100 parts by weight of the antibody or antibody mixture.

7. The mass spectrometric method of claim 1, wherein the extracting is carried out at 4 to 25° C.
